(19) United States
(12) Patent Application Publication
(10) Pub. No.: US 2023/0240609 A1
(43) Pub. Date: Aug. 3, 2023

(54) IN-EAR OPTICAL SENSORS FOR AR/VR APPLICATIONS AND DEVICES

(71) Applicant: Meta Platforms Technologies, LLC, Menlo Park, CA (US)

(72) Inventors: Morteza Khaleghimeybodi, Bothell, WA (US); Barry David Silverstein, Kirkland, WA (US); Nils Thomas Fritiof Lunner, Redmond, WA (US); John Rumsfeld, San Francisco, CA (US); Andrew John Ouderkirk, Kirkland, WA (US); Joshua Weiner, Great Neck, NY (US)

(21) Appl. No.: 18/069,045

(22) Filed: Dec. 20, 2022

Related U.S. Application Data
(60) Provisional application No. 63/305,932, filed on Feb. 2, 2022.
(60) Provisional application No. 63/356,851, filed on Jun. 29, 2022.
(60) Provisional application No. 63/356,860, filed on Jun. 29, 2022.
(60) Provisional application No. 63/356,864, filed on Jun. 29, 2022.
(60) Provisional application No. 63/356,872, filed on Jun. 29, 2022.
(60) Provisional application No. 63/356,877, filed on Jun. 29, 2022.
(60) Provisional application No. 63/356,883, filed on Jun. 29, 2022.

(51) Int. Cl. A61B 5/00; A61B 5/145; A61B 5/1455; G02B 27/01

(52) U.S. Cl. CPC A61B 5/6817; A61B 5/14532; A61B 5/7246; A61B 5/7228; A61B 5/14551; G02B 27/0172; G06F 3/015

(57) ABSTRACT

An in-ear device for immersive reality applications is provided. The device includes a fixture configured to fit in an ear canal of a user, an emitter mounted on the in-ear fixture and configured to emit a first electromagnetic radiation onto the ear canal of the user, a detector configured to provide a signal indicative of a second electromagnetic radiation from the ear canal of the user, and a processor that is coupled to an augmented reality headset, the processor configured to identify a health condition of the user based on the signal, wherein the second electromagnetic radiation includes at least a portion of the first electromagnetic radiation reflected from a tissue in the ear canal of the user. A memory storing instructions to cause processors in a system to perform a method for use of the above in-ear device, the memory, the processor, the system and the method are also provided.

### CROSS-REFERENCE TO RELATED APPLICATIONS

The present disclosure is related and claims priority under 35 U.S.C. § 119(e) to U.S. Prov. Appln. No. 63/305,932, entitled IN-EAR BIO-SENSING FOR AR/VR APPLICATIONS AND DEVICES, filed on Feb. 2, 2022, to U.S. Prov. Appln. No. 63/356,851, entitled IN-EAR ELECTRODES FOR AR/VR APPLICATIONS AND DEVICES, to U.S. Prov. Appln. No. 63/356,860, entitled IN-EAR OPTICAL SENSORS FOR AR/VR APPLICATIONS AND DEVICES, to U.S. Prov. Appln. No. 63/356,864, entitled IN-EAR MOTION SENSORS FOR AR/VR APPLICATIONS AND DEVICES, to U.S. Prov. Appln. No. 63/356,872, entitled IN-EAR TEMPERATURE SENSORS FOR AR/VR APPLICATIONS AND DEVICES, to U.S. Prov. Appln. No. 63/356,877, entitled IN-EAR MICROPHONES FOR AR/VR APPLICATIONS AND DEVICES, to U.S. Prov. Appln. No. 63/356,883, entitled IN-EAR SENSORS AND METHODS OF USE THEREOF FOR AR/VR APPLICATIONS AND DEVICES, all filed on Jun. 29, 2022, to Morteza KHALEGHIMEYBODI, et al., the contents of which applications are hereby incorporated by reference in their entirety, for all purposes.

### BACKGROUND

### Field

The present disclosure is related to in-ear optical sensors for use in virtual reality and augmented reality environments and devices. More specifically, the present disclosure is related to optical sensors configured to monitor the volume and walls in the ear canal for health assessment of users of in-ear devices for immersive reality applications.

### Related Art

Current in-ear devices (e.g., hearing aids, hearables, headphones, earbuds, and the like) for mobile and immersive applications are typically bulky and uncomfortable for the user. Accordingly, adding optical sensors to in-ear devices is hindered by the small form factors desirable in such devices and the complex data processing and analysis involved, in addition to the processing and memory capabilities desired in such devices.

### SUMMARY

In a first embodiment, a device includes an in-ear fixture configured to fit in an ear canal of a user, an emitter mounted on the in-ear fixture and configured to emit a first electromagnetic radiation onto the ear canal of the user, a detector configured to provide a signal indicative of a second electromagnetic radiation from the ear canal of the user, and a processor that is coupled to an augmented reality headset, the processor configured to identify a health condition of the user based on the signal, wherein the second electromagnetic radiation includes at least a portion of the first electromagnetic radiation reflected from a tissue in the ear canal of the user.

In a second embodiment, a system includes a memory storing multiple instructions, and one or more processors configured to execute the instructions and cause the system to perform operations. The operations include to transmit, into an ear canal of a user of an in-ear device, a first electromagnetic radiation, to receive, from an electromagnetic detector, a signal indicative of a second electromagnetic radiation responsive to the first electromagnetic radiation, and to identify a health condition of the user based on a difference between the first electromagnetic radiation and the second electromagnetic radiation. In addition to the above bio-sensing operations, other common audio signal processing operations such as signal processing instruction for performing active noise cancelation, transparent hear-through audio filter, occlusion mitigation, and the like are also part of the system operations.

In a third embodiment, a computer-implemented method includes transmitting, into an ear canal of a user of an in-ear device, a first electromagnetic radiation, receiving, from an electromagnetic detector, a signal indicative of a second electromagnetic radiation responsive to the first electromagnetic radiation, and identifying a health condition of the user based on a difference between the first electromagnetic radiation and the second electromagnetic radiation.

In other embodiments, a non-transitory, computer-readable medium stores instructions which, when executed by a processor, cause a computer to perform a method. The method includes transmitting, into an ear canal of a user of an in-ear device, a first electromagnetic radiation, receiving, from an electromagnetic detector, a signal indicative of a second electromagnetic radiation responsive to the first electromagnetic radiation, and identifying a health condition of the user based on a difference between the first electromagnetic radiation and the second electromagnetic radiation.

In yet other embodiments, a system includes a first means to store instructions, and a second means to execute the instructions to cause the system to perform a method. The method includes transmitting, into an ear canal of a user of an in-ear device, a first electromagnetic radiation, receiving, from an electromagnetic detector, a signal indicative of a second electromagnetic radiation responsive to the first electromagnetic radiation, and identifying a health condition of the user based on a difference between the first electromagnetic radiation and the second electromagnetic radiation.

These and other embodiments will become clear to one of ordinary skills, in view of the following.

### BRIEF DESCRIPTION OF THE FIGURES

FIG. 1 illustrates an AR headset and an in-ear monitor (IEM) in an architecture configured to assess a user's health, according to some embodiments.

FIG. 2 illustrates an augmented reality ecosystem including wearable devices in the ear and wrist to assess a user's health, according to some embodiments.

FIGS. 3A-3D illustrate different embodiments of an in-ear monitor (IEM), according to some embodiments.

FIGS. 4A-4B illustrate photoplethysmography (PPG) sensors in an IEM, according to some embodiments.

FIG. 5 illustrates a waveform provided by a PPG sensor in an IEM, according to some embodiments.

FIG. 6 illustrates an infrared spectrum indicative of a glucose measurement in an optical sensor for an IEM, according to some embodiments.

FIGS. 7A-7F illustrate several embodiments for a bio-measurement sensor in an IEM based on surface plasmon resonance, according to some embodiments.

FIGS. 8A-8C illustrate several embodiments for a chemical measurement sensor in an IEM based on surface plasmon resonance, according to some embodiments.

FIG. 9 is a flow chart illustrating steps in a method for using optical sensors in an in-ear monitor for assessing the health of a user of a headset or smart glasses, according to some embodiments.

FIG. 10 is a block diagram illustrating an exemplary computer system with which headsets and other client devices, and the method in FIG. 9 be implemented, according to some embodiments.

In the figures, elements having the same or similar reference numeral have the same or similar features and attributes, unless explicitly stated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

General Overview

Head-worn devices (e.g., devices worn on head including but not limited to hearables, glasses, AR/VR headsets and smart glasses, etc.) offer opportunities to access valuable health information.

The ear (e.g., the ear canal and ear concha and pinna) has close proximity to the brain, to body chemistry, and blood vessels indicative of brain activity and cardio-respiratory activity, and inner body temperature. More specifically, sensors including electrodes, inertial motion units (IMUs), accelerometers, and microphones can be placed inside the ear canal or around the ear (in the case of AR/VR headsets or smart glasses) to sense brain, heart, and electrophysiological activities (e.g., electro-encephalography EEG, electro-cardiography ECG, electro-oculography, EOG, electrodermal activity, EDA, and the like); or to sense vital signs (heart rate, breathing rates, blood pressure, body temperature, and the like); or to sense the body chemistry (e.g., blood alcohol level, blood glucose estimation, and the like).

Microphones as disclosed herein may include contact microphones to detect motion, internal microphones and external microphones, acoustic microphones, and the like. In addition to microphones, in-ear devices as disclosed herein may also include speakers to generate and provide sound signals to the user of the in-ear device.

Electrodes in embodiments as disclosed herein may be used in EOG, ECG, and EEG measurements, e.g., for determining auditory attention, heart rate estimation, breathing rate, and the like, Auditory Steady State Response—ASSR—, auditory brainstem response—ABR—. In some embodiments, in-ear electrodes as disclosed herein may be useful to measure resting state electric oscillations (alpha waves in an EEG) that can track relaxation/activity. With the combination of other measurements (e.g., photoplethysmography, PPG), a new branch of diagnostic possibilities is open. In-ear EEG measurements can be applied to track user attention (e.g., distinguishing between attention focus from eye gaze direction).

Methods and devices disclosed herein include optical, acoustical, motion sensors, chemical sensors, and temperature sensors, in and around the ears of AR/VR headset users, in combination with software correlation of the signals provided by the above sensors to generate comprehensive diagnostics and health evaluation of the user.

Some of the features disclosed herein include in-ear or head-worn body temperature sensing using infrared sensing and spectroscopy techniques. In some embodiments, the contact area for sensors as disclosed herein include the in-ear canal (like an in-ear earbud) and within the conchal bowl (in human pinna), areas on top of the human ear (where the glasses sit), and areas in the nose-pad of a headset or smart glasses (where glasses sit on the nose). Some measurements may include in-ear or around the ear sensing of glucose level, alcohol sensing, body temperature, blood pressure, and the like. Some embodiments include pulse transit time (PTT) methodology to estimate blood pressure for a glasses/headset device using a combination of optical and electrical signals (e.g., PPG+ECG sensors respectively). Some embodiments obtain user's blood pressure using an optical sensing technique (PPG) in combination with a deep neural network to train a network based using both PPG information and a corresponding ground-truth blood pressure information. Some embodiments obtain user's blood pressure using an optical sensing technique with multiple distinct optical wavelengths and using a technique called multi-wavelength pulse transit time photoplethysmography (MWPTT PPG) in combination with a deep neural network to train a network based using both PPG information and a corresponding ground-truth blood pressure information. Some embodiments include motion-based pulse transit time (PTT) methodology to estimate blood pressure for a glass/headset device using a combination of motion sensor and electrical signals (e.g., IMU+ECG sensors respectively). Once fully trained, the neural network can then quantify and predict the user's blood pressure using just the PPG information and leveraging this pre-trained network. To further improve the accuracy, some subjective calibrations may be desirable. In some embodiments, PPG signals collected in IEM devices as disclosed herein may be able to estimate the cognitive load on the user with analysis of oxygenated and deoxygenated blood flow (oxy- and deoxy-hemoglobin) to the brain. Some embodiments include sensing alcohol levels through emissions around the ear. Some embodiments incorporate chemical sensing intake around the contact points of the ear. In some embodiments, IEM devices may perform alcohol monitoring and fat burning during user exercise.

### Example System Architecture

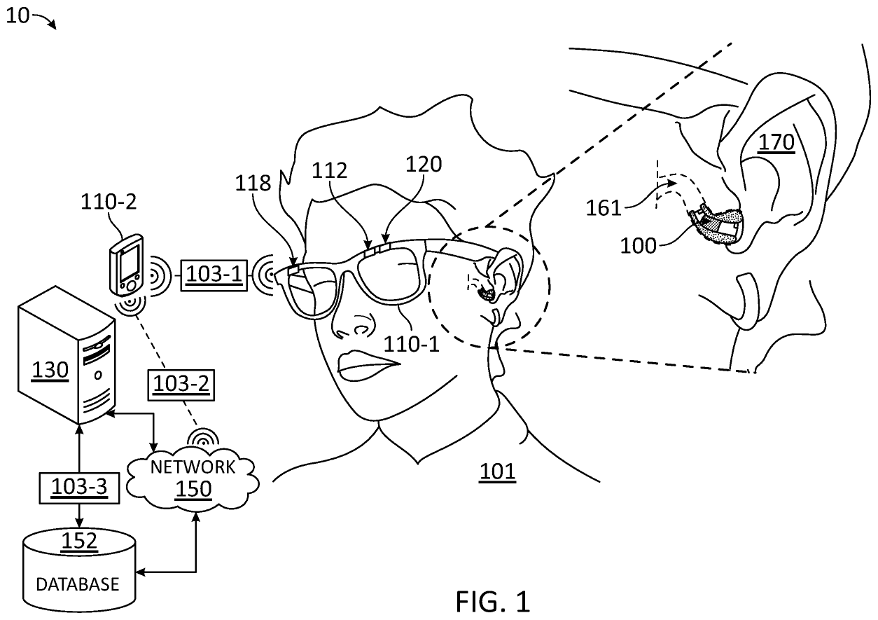

FIG. 1 illustrates an AR headset 110-1 and an in-ear monitor (IEM) 100 in an architecture 10 configured to assess the health of a user 101, according to some embodiments. IEM 100 is inserted in the ear 170 of user 101, reaching the ear canal 161. AR headset 110-1 may include smart glasses having a memory circuit 120 storing instructions and a processor circuit 112 configured to execute the instructions to perform steps as in methods disclosed herein. AR headset 110-1 (or smart glasses) may also include a communications module 118 configured to wirelessly transmit information (e.g., Dataset 103-1) between AR headset 110-1 (and/or in-ear device 100, and/or a smart watch, or combination of the above) and a mobile device 110-2 with the user (AR headset 110-1 and mobile device 110-2 will be collectively referred to, hereinafter, as “client devices 110”). Communications module 118 may be configured to interface with a network 150 to send and receive information, such as dataset 103-1, dataset 103-2, and dataset 103-3, requests, responses, and commands to other devices on network 150. In some embodiments, communications module 118 can include, for example, modems or Ethernet cards. Client devices 110 may in turn be communicatively coupled with a remote server 130 and a database 152, through network 150, and transmit/share information, files, and the like with one another (e.g., dataset 103-2 and dataset 103-3). Datasets 103-1, 103-2, and 103-3 will be collectively referred to, hereinafter, as “datasets 103.” Network 150 may include, for example, any one or more of a local area network (LAN), a wide area network (WAN), the Internet, and the like. Further, the network can include, but is not limited to, any one or more of the following network topologies, including a bus network, a star network, a ring network, a mesh network, a star-bus network, tree or hierarchical network, and the like.

In some embodiments, at least one of the steps in methods as disclosed herein are performed by processor 112, providing dataset 103-1 to mobile device 110-2. Mobile device 110-2 may further process the signals and provide dataset 103-2 to database 152 via network 150. Remote server 130 may collect dataset 103-2 from multiple AR headsets 110-1 and mobile devices 110-2 in the form and perform further calculations. In addition, having aggregated data from a population of individuals, the remote server may perform meaningful statistics. This data cycle may be established provided each of the users involved have consented for the use of de-personalized, or anonymized data. In some embodiments, remote server 130 and database 152 may be hosted by a healthcare network, or a healthcare facility or institution (e.g., hospital, university, government institution, clinic, health insurance network, and the like). Mobile device 110-2, AR headset 110-1, in-ear device 100, and applications therein may be hosted by a different service provider (e.g., a network carrier, an application developer, and the like). Moreover, AR headset 110-1 and mobile devices 110-2 and in-ear device 100 may proceed from different manufacturers. User 101 is ultimately the sole owner of dataset 103-1 and all data derived therefrom (e.g., datasets 103), and so all the data flows (e.g., datasets 103), while provided, handled, or regulated by different entities, are authorized by user 101, and protected by network 150, server 130, database 152, and mobile device 110-2 for privacy and security.

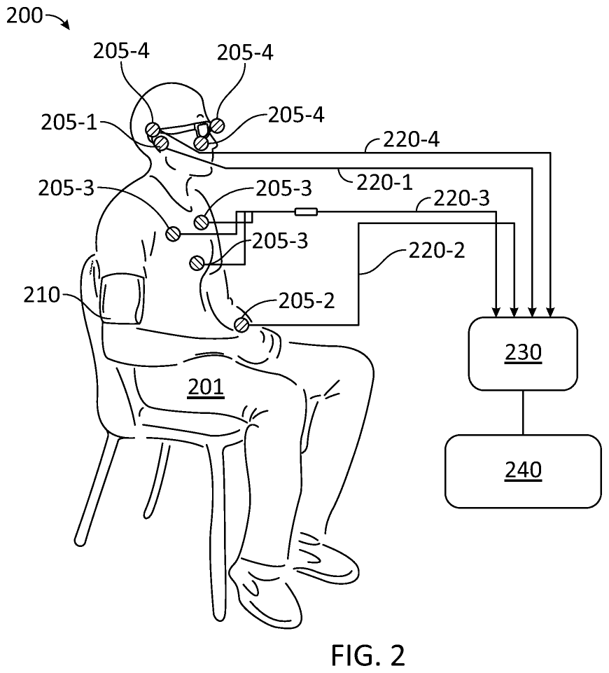

FIG. 2 illustrates an augmented reality ecosystem 200 including wearable devices in the ear 205-1 (e.g., an IEM), wrist 205-2, chest 205-3, and smart glass sensors 205-4 to assess the health of user 201, according to some embodiments. In some embodiments, IEM 205-1 further includes an optical sensor configured to provide an optical signal 220-1 to a processor in a computer 240 via a data acquisition module (DAQ) 230. IEM 205-1 may further include one or more contact electrodes configured to provide an electrical signal to a processor in a computer 240 via a data acquisition module (DAQ) 230. Computer 240 is configured to identify a cardiovascular condition of user 201 based on a first electronic signal from IEM 205-1 and optical signal 220-1. In some embodiments, IEM 205-1 further includes a motion sensor (e.g., an accelerometer, a contact microphone, or an IMU) configured to provide a motion-based signal to computer 240 via DAQ 230. In some embodiments, a pair of IEMs 205 will be placed in both ears and different optical, electrical (electrode), acoustic (microphone), or motion sensors (accelerometer, IMU, contact microphone, etc.) may be placed in either both sides; or in some cases, some sensors may be placed on one side (e.g., the Right side) and some other sensors may be placed on the other side (e.g., Left side). Computer 240 is configured to identify a cardiovascular condition of the user based on a first electronic signal from IEM 205-1 and the motion signal. The optical sensor may be a photo-plethysmography (PPG) sensor and optical signal 220-1 may include a digital or analog signal indicative of a vascular activity inside the ear of user 201. Chest sensors 205-3 and smart glass sensors 205-4 may include ECG sensors to provide a distributed signals 220-3 and 220-4 from one or more areas around the chest and face (e.g., the outside of the ear, the chin, and the nose) of user 201, respectively (or alternatively an ECG can be collected from some electrodes placed on areas on the head or from electrodes placed in IEM 205-1, or electrodes placed on the wrist device 205-2), and a wrist PPG sensor in device 205-2 may provide a separate signal 220-2 for vascular activity around the wrist of user 201. IEM 205-1, wrist sensor 205-2, chest sensors 205-3, and smart glass sensors 205-4 will be collectively referred to, hereinafter, as “wearable devices (and sensors) 205.” Blood pressure (BP) measurements may be obtained with a cuff or cuff-less BP monitor 210 and may also be determined by comparing PPG signals 220-1 and 220-2. Signals 220-1, 220-2, 220-3 and 220-4 (hereinafter, collectively referred to, hereinafter, as “signals 220”) may be collected and digitized by DAQ 230 in computer 240, for processing. In some embodiments, signals 220 and others may be wired, or wireless. In some embodiments, it may be preferable to have wireless signal communication between the different wearable devices 205 with user 201. In some embodiments, wearable devices and sensors 205 may include one or more motion sensors, and the motion-based information collected from the smart glass, the IEM, chest or wrist can be combined to create a more meaningful information.

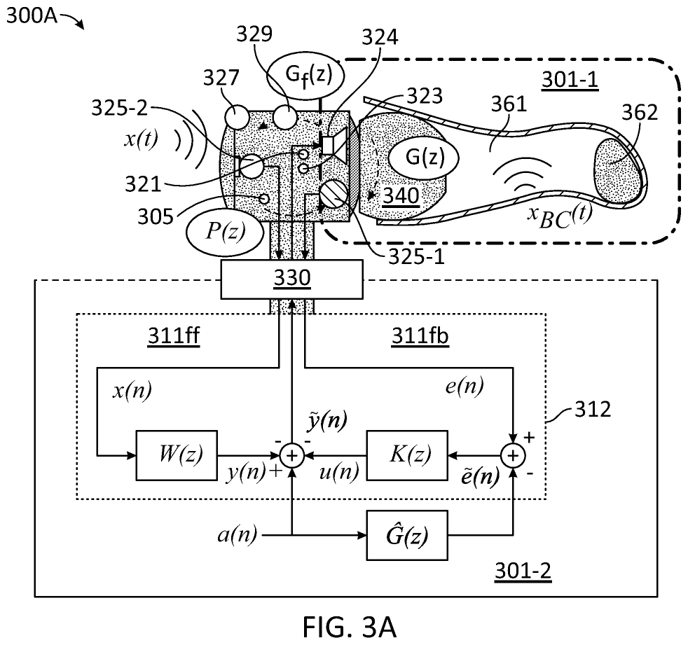

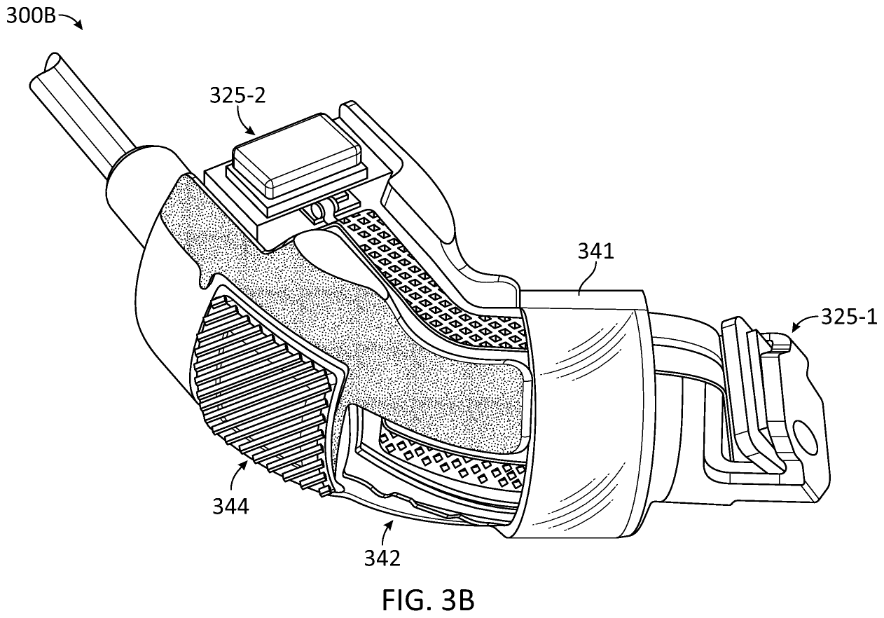

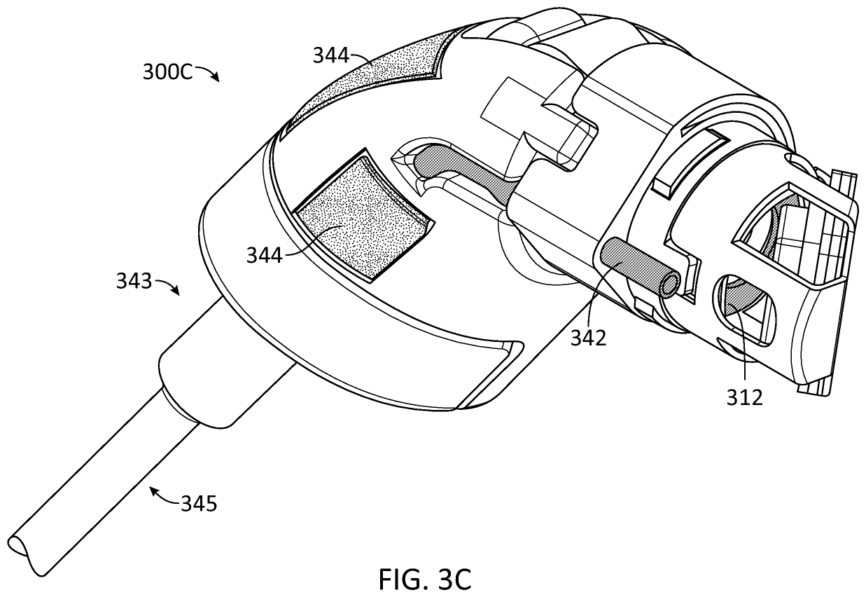

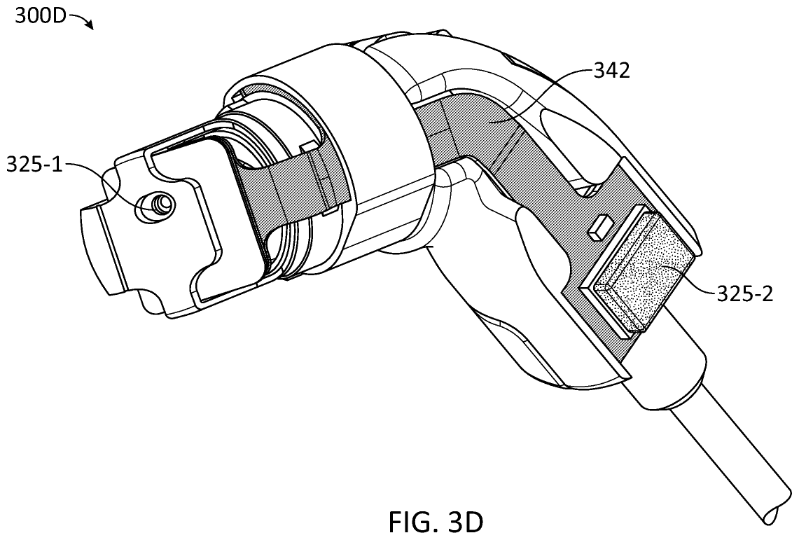

FIGS. 3A-3D illustrate different embodiments of an in-ear monitor (IEM) 300A, 300B, 300C, and 300D (hereinafter, collectively referred to as “IEMs 300”), according to some embodiments. IEMs 300 may include a front end 301-1 including sensors and open to ear canal 361 and ear drum 362, and a back end 301-2 including a processor 312. IEMs 300 may include sensors such as: one or more electrodes 305 to sense electrical signals, acoustic sensors 325-1 and 325-2 (e.g., collectively referred hereinafter, as “microphones 325”), motion sensors 327 (e.g., accelerometers, contact microphones, inertial motion units—IMUs, and the like), temperature sensors 329, and optical sensors including one or more emitters 321 and one or more detectors 323 (e.g., LEDs and PDs in PPG sensors, functional near-infrared fNIR sensors—Fourier transform based, spectroscopic based-). Electrodes 305 may include bio-potential electrodes for applications such as EEG, ECG, EOG, and EDA). In addition, processor 312 may handle at least some of the operations for signal acquisition and control of components and sensors 321, 323, 324 (a speaker), 325-1 (internal microphone), 325-2 (external microphone, hereinafter, collectively referred to as “microphones 325”), 327, and 329 via a digital-to-analog and/or analog-to-digital converter (DAC/ADC) 330. Processor 312 may include a feedforward stage 311ff and a feedback stage 311fb that cooperate to process the signal from the sensors: noise reduction, balancing, filtering, and amplification.

In some embodiments, electrodes 305 include a contact electrode configured to transmit a current from the skin in the ear canal of the user. In some embodiments, an electrode 305 is coated with at least one of a gold layer, a silver layer, a silver chloride layer, or a combination thereof. In some embodiments, electrodes 305 include a capacitive coupling electrode disposed sufficiently close, but not in contact, with the user's skin. In some embodiments, IEMs 300 further include at least a second electrode 305 mounted on in-ear fixture 340, the second electrode 305 configured to receive a second electronic signal from the skin in ear canal 361. In some embodiments, processor 312 is configured to select the first electronic signal when a quality of the first electronic signal is higher than a pre-selected threshold. In some embodiments, processor 312 is configured to reduce a noise background from the first electronic signal with the second electronic signal. In some embodiments, processor 312 is configured to determine a heart rate of the user from the first electronic signal. In some embodiments, processor 312 is configured to determine a brain activity from the first electronic signal that corresponds to an acoustic stimulus received in the external microphone.

In some embodiments, emitter 321 is configured to emit a first electromagnetic radiation onto ear canal 361. Accordingly, detector 323 is configured to provide a signal indicative of a second electromagnetic radiation from the ear canal of the user, wherein the second electromagnetic radiation includes at least a portion of the first electromagnetic radiation reflected from a tissue in ear canal 361. In some embodiments, the first electromagnetic radiation includes one of a near-infrared or red or green light (or any other optical wavelength within visible and near infrared or infrared range), and the health condition of the user includes a cardio-respiratory condition. In some embodiments, a difference between the first electromagnetic radiation and the second electromagnetic radiation is indicative of a trace amount of a selected molecule in the air filling ear canal 361. For example, in some embodiments (e.g., absorption spectroscopy), the difference between the first electromagnetic radiation and the second electromagnetic radiation is a portion of the first electromagnetic radiation absorbed by the selected molecule. Accordingly, the selected molecule may have a strong absorption spectrum within the spectral bandwidth of the first electromagnetic radiation. In some embodiments, the second electromagnetic radiation may include a portion of the first electromagnetic radiation that is scattered back to the detector by the skin, or the blood in a blood vessel in the ear canal of the user. Accordingly, the difference between the first and second electromagnetic radiation may be inversely related to the amount of blood or volume of a blood vessel (e.g., a larger amount of second electronic radiation being associated with a bigger blood vessel containing more blood in a systolic period of a heart cycle, as in PPG). In some embodiments, the first electromagnetic radiation is a stimulating radiation to generate the second electromagnetic radiation, such as a Raman radiation or fluorescence radiation. Accordingly, in some embodiments, an optical sensor as disclosed herein may include at least one filter to block a selected portion of the spectral bandwidth of the first or second electromagnetic radiation. In some embodiments, detector 323 may be an intensity-based detector; in some other embodiments, detector 323 may be a spectrometer-based detector. For example, an array of detectors 323 with variable spectral sensitivity can be used to detect the spectral contents of the reflected second electromagnetic radiation.

In some embodiments, the first electromagnetic radiation may be directed to different areas of the ear, and the second electromagnetic radiation may be selected to provide the best signal-to-noise ratio. Accordingly, in some embodiments, emitter 321, detector 323, or both, may be directed to different areas of the user's ear to find areas with the highest signal-to-noise ratio. To achieve this, a mirror (e.g., micro-electromechanical system—MEMS), a pancake lens or pancake wedge lens. or a liquid lens with an adjustable prism may selectively adjust the orientation of light generated by, or received in, emitter 321 or detector 323.

In some embodiments, emitter 321 and detector 323 may be part of a self-mixing interferometer (SMI). An SMI is a compact, low power, inexpensive and sensitive optical device configured to measure reflectivity, including back scatter, as well as displacement of the skin. In some embodiments, a displacement of the skin obtained with an SMI is combined with heart rate measurements (e.g., from PPG sensors, motion sensors or ECG electrodes) to measure blood pressure and heart rate, or even vibration of the eardrum to also act as an internal microphone.

IEMs 300 in the AR headset or smart glasses may include an in-ear fixture 340 configured to hermetically seal an ear canal of a user, a first electrode 305 mounted on in-ear fixture 340 and configured to receive a first electronic signal from a skin in ear canal 361, and an internal microphone 325-1 coupled to receive an internal acoustic signal, propagating through ear canal 361. An acoustic front end includes internal microphone 325-1 configured to detect acoustic waves (x_{BC}(t)) propagated through ear canal 361 and generated by the inner body (e.g., heart rate at about ≤100 Hz, breathing rate at about 50-1000 Hz, and other sounds in the laryngeal cavity). An external microphone 325-2 is coupled to receive an external acoustic signal x(t), propagating through an environment of the user. In some embodiments, the internal signal x_{BC}(t) in conjunction with the external signal x(t) may be used in acoustic procedures such as audio streaming, hear-through, active noise cancelation (ANC), hearing corrections, virtual presence and spatial audio, call services, and the like. In some embodiments, at least some of the above processes are performed in conjunction between left-ear and right-ear IEM monitors 300.

IEM 300B includes a sealing gasket 341 that separates the inner portion of ear canal 361 from the environment, leaving a back-volume vent including an acoustically resistive mesh 344 for a pressure equalizer (PEQ) tube 342 to vent into resistive mesh 344 (also shown in IEM 300C). The sealed cavity may enable breathing and heart rate monitoring (e.g., isolating the signal from internal acoustic microphone 325-1) at low power usage and with a small form factor.

IEM 300C illustrates processor circuit 312 to identify a cardiovascular condition or a neurologic condition of the user, based on at least one of a first electronic signal, an internal acoustic signal, and an external acoustic signal (e.g., from microphones 325). Some embodiments may include a down cable 345 to electrically couple the IEM with the VR headset or smart glasses, including a strain relief 343.

IEM 300D illustrates a flexible, printed circuit board (FPCB) 342 that provides internal electrical connectivity to the different components and sensors 321, 323, 325, 327, and 329.

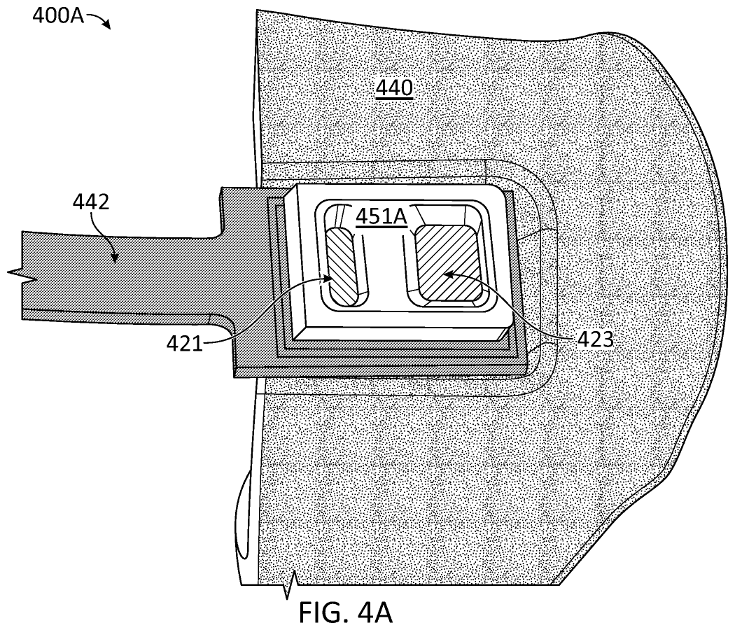

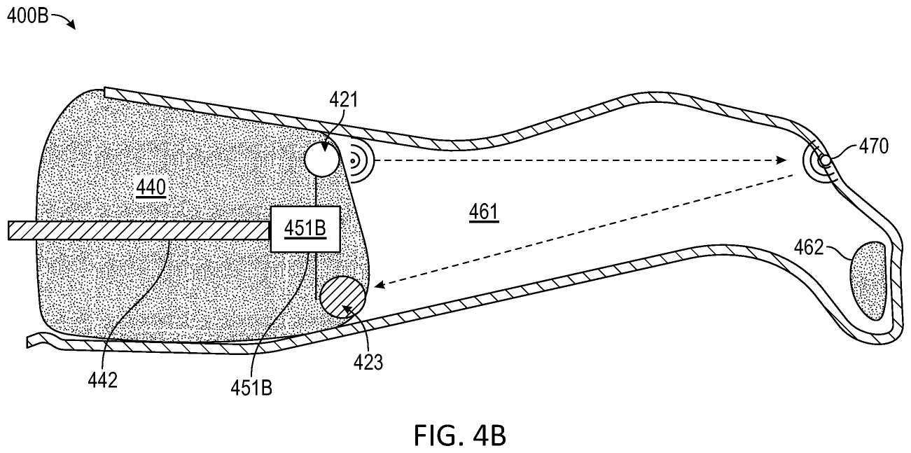

FIGS. 4A-4B illustrate PPG sensors 451A and 451B (hereinafter, collectively referred to as “PPG sensors 451”) in IEMs 400A and 400B (hereinafter, collectively referred to as “IEMs 400”), according to some embodiments. PPG sensors 451 may include an emitter 421 and a detector 423 mounted on an in-ear fixture 440 of IEMs 400. A flex connector 442 provides power for emitter 421 and collects the signal from detector 423 to a processor for data filtering, analysis, and measurement.

PPG sensor 451A is a contact sensor, wherein emitter 421 and detector 423 are placed adjacent to one another and facing a side of in-ear fixture 440, which is in close proximity or in contact with the skin inside the ear canal (cf. ear canal 161 or 361). Accordingly, a first electromagnetic radiation from emitter 421 interacts with blood vessels in the skin and is scattered into a second electromagnetic radiation, back to detector 423. The higher the amount of blood and volume of the blood vessel, the larger the amount of second electromagnetic radiation expected at detector 423. Emitter 421 may generate light at one or more than one distinct wavelengths. In some embodiments, emitter 421 may generate three (3) distinct wavelengths or colors (e.g., red, blue, and infrared) that may further be either synchronously or asynchronously turned ‘on,’ to enable different health sensing capabilities.

PPG sensor 451B is a remote sensor, wherein emitter 421 projects a beam onto a remote spot 470 within ear canal 461. In some embodiments, spot 470 may be selected within the eardrum 462. The reflected signal is collected by sensor 423. Emitter 421 may include a monochromatic infrared, red, green, or blue light, or a combination thereof. Sensor 423 measures the reflectivity of the skin on spot 470, which is modulated according to a heart rate of the user. Sensor 423 may include a single photodetector, a linear array, or a 2D array of, for example, CMOS or CCD elements. The detectors in sensor 423 may include broadband sensors, or different sensors configured to measure different wavelengths of light from emitter 421 (e.g., to measure blood oxygenation, and the like). A 2D array may provide an RGB image of spot 470, so that an accurate HR determination can be made. During each heartbeat, changes in blood volume cause regulated light transmission and reflection, contributing to subtle skin color changes that are invisible to the naked eye but can be captured by a 2D sensor array 423. In practical scenarios, in-ear channel 361 may be spotted by sensor 423 as the region-of-interest because the skin is relatively thin and close to blood vessels, thus possessing positive measuring performance. The same is true for eardrum 462.

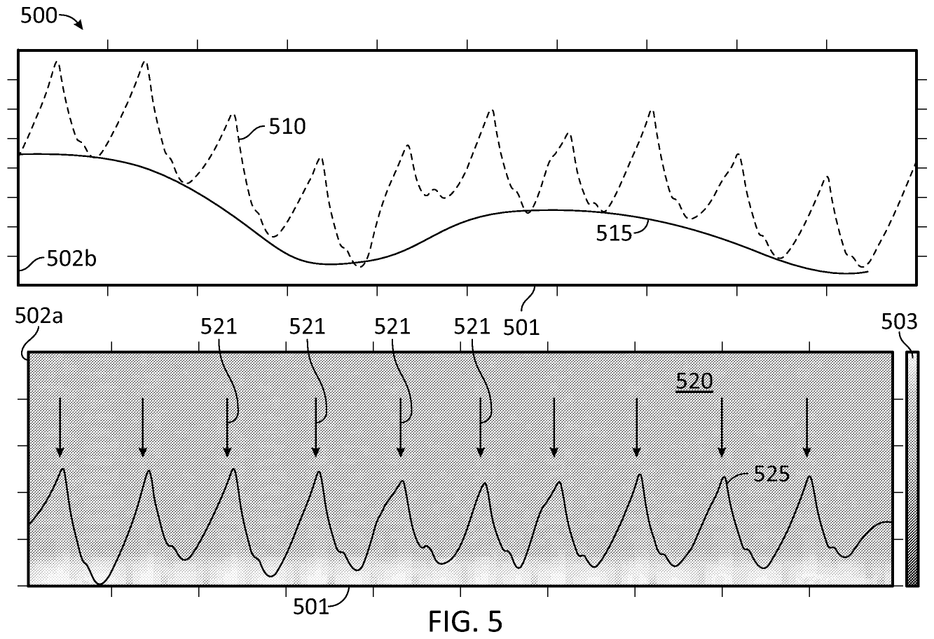

FIG. 5 is a chart 500 illustrating a waveform 510 and a frequency spectrum 520 provided by a PPG sensor in an IEM, according to some embodiments. The abscissae 501 in chart 500 indicates time (e.g., seconds), ordinates 502a indicate frequency (e.g., Hertz), and ordinates 502b indicate signal amplitude (e.g., units, counts, millivolt, etc.). A grayscale 503 indicates power spectral density. Waveform 510 and spectrum 520 clearly indicate the heart pulses 521 (e.g., peaks corresponding to systolic compressions). In addition, PPG waveform 510 also includes a lower frequency modulation 515 associated with a breathing rate of the user.

In some embodiments, spectral power decomposition 520 of PPG waveform 510 identifies and disentangles different components in the PPG signal. Spectral power decomposition 520 may be obtained by a processor in the IEM (cf. processor 312), or by a processor in a mobile device or in a remotely coupled server (e.g., processor 112, client devices 110, and server 130), in real time or asynchronously. A profile of the low frequency components 525 follows closely the heartbeats in waveform 510.

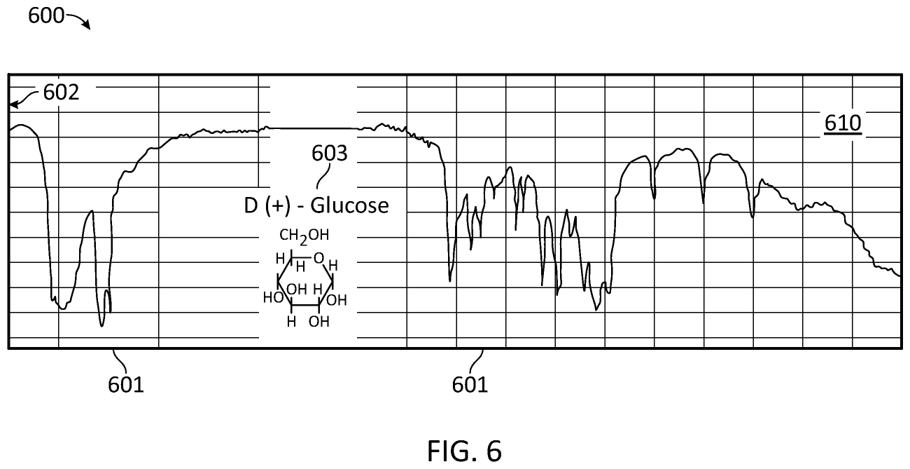

FIG. 6 is a chart 600 illustrating an infrared spectrum 610 indicative of a glucose absorption measurement in an optical sensor for an IEM, according to some embodiments. The abscissae in chart 600 may be wavelength 601 (e.g., in microns μm), and the ordinates may indicate transmittance 602 (in normalized units). Accordingly, an emitter in an IEM as disclosed herein may have a spectral bandwidth within the areas of the absorption spectrum of glucose 603 that have a low transmittance (e.g., at a wavelength around 3, 3.5, 7, 9, and 9.5-10 μm). Accordingly, a difference between a first electromagnetic radiation emitted and a second electromagnetic radiation transmitted through, or reflected from, a sample, is indicative of a content of glucose 603 in the user's bloodstream.

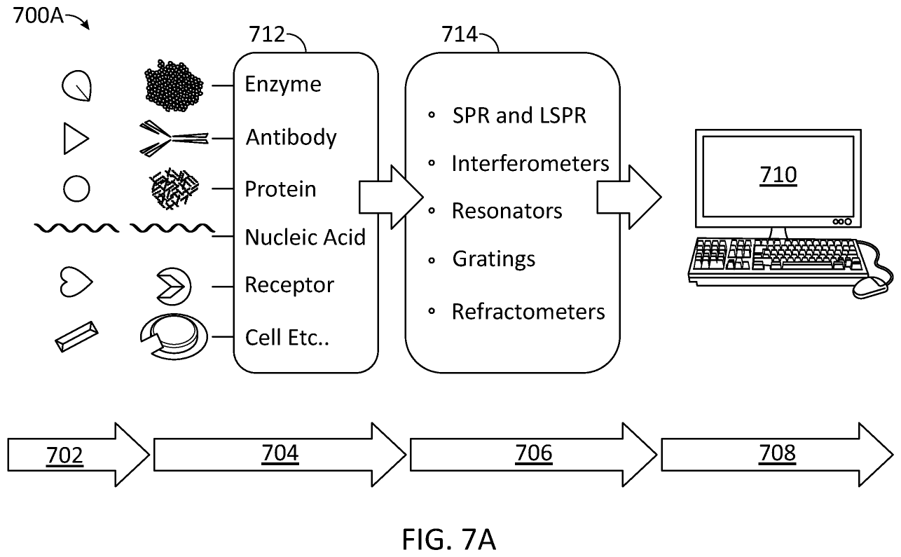

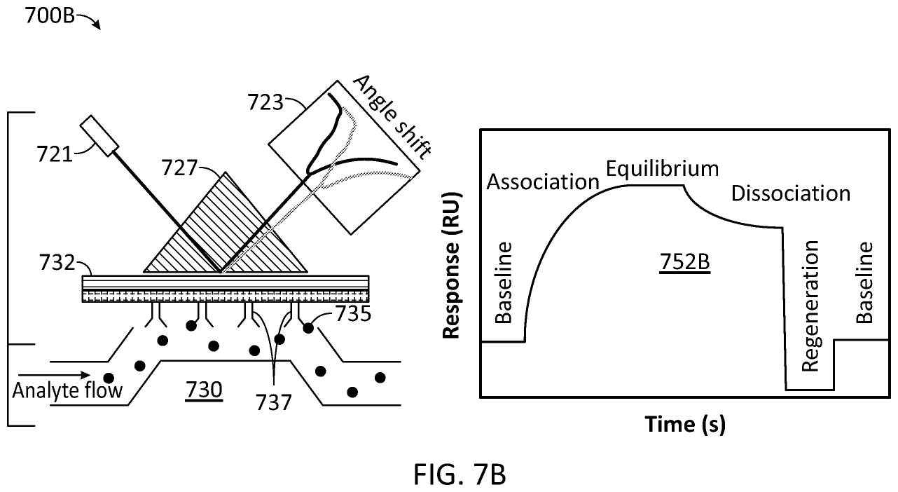

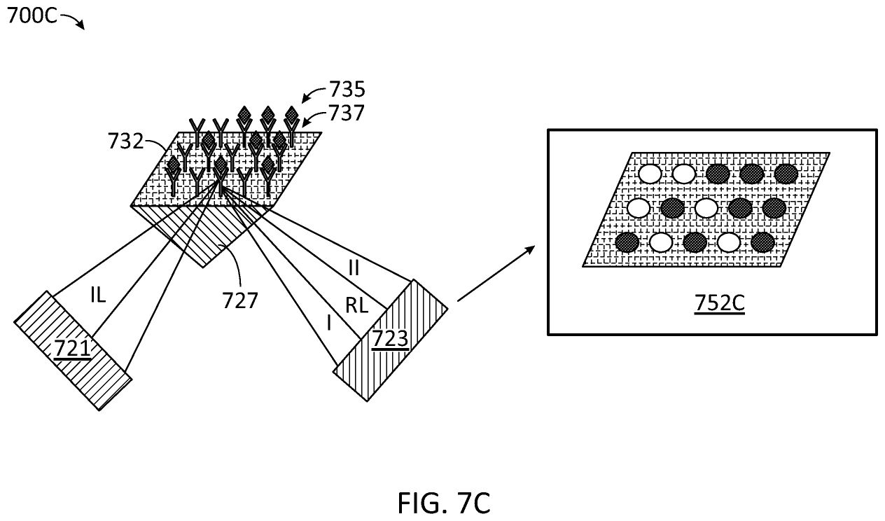

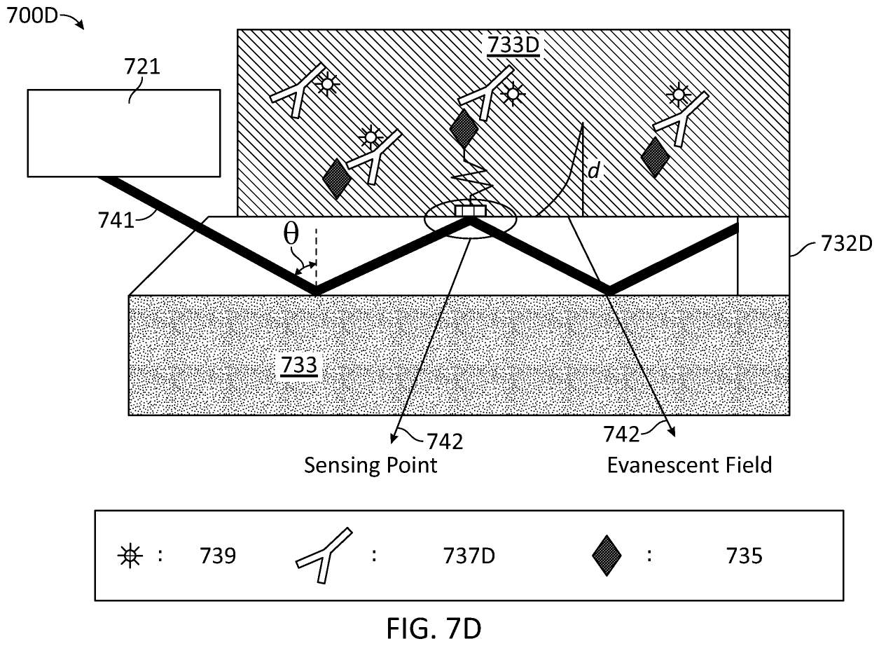

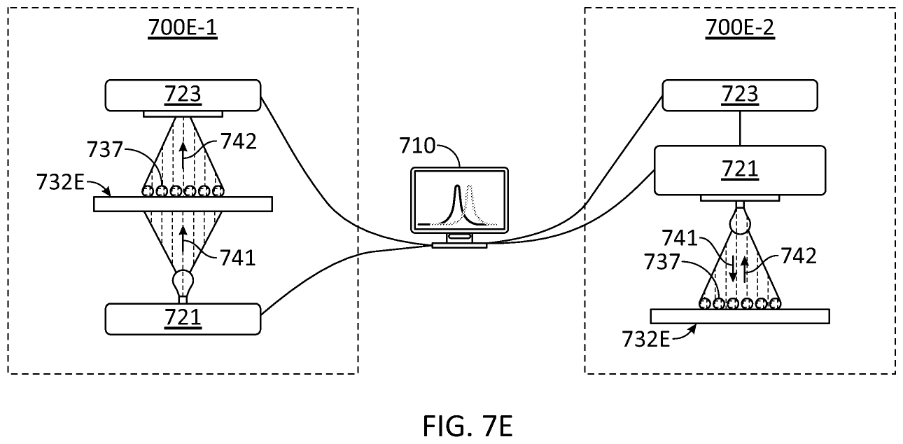

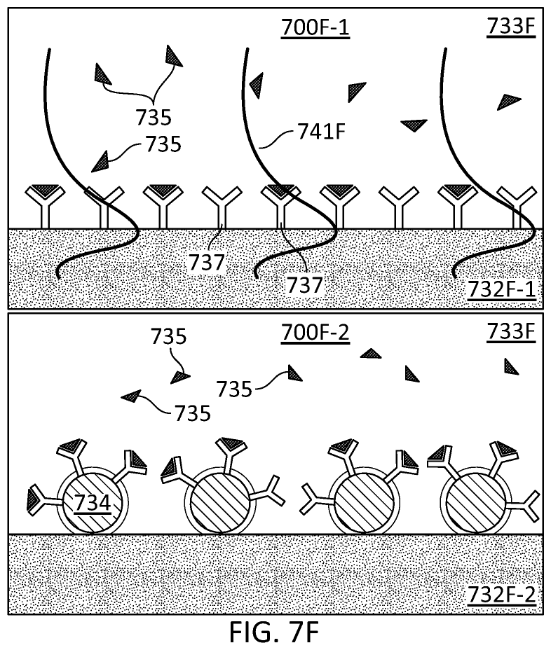

FIGS. 7A-7F illustrate several embodiments for bio-measurement sensors 700A, 700B, 700C, 700D, 700E-1, 700E-2 (“700E”), and 700E-1 and 700E-2 (700F, hereinafter, all collectively referred to as “bio-measurement sensors 700”) in an IEM based on surface plasmon resonance, according to some embodiments. In some embodiments, an IEM device may include a chip 732 having a functional layer of molecules 737 that are optically active and chemically sensitive to selected target analytes 735 (e.g., a pathogen such as a virus, a bacterium, a DNA string, a protein, or even a small molecule, inorganic, organic, organometallic or heteroatom molecules, or enzymes, antibodies, and combinations thereof). Accordingly, an electromagnetic radiation measured at a detector 723 (e.g., photodetector or spectrometer) may be indicative of a change in the optical property of the functional layer of molecules 737, which may be proportionally or otherwise associated with a precise amount of selected target analyte 735 in the sample. A computer 710 may process the analysis,

In some embodiments, the IEM may include a chip 732 having a metallic layer configured to form a plasmon resonance in response to an electromagnetic radiation provided by an emitter 721. Emitter 721 may be a broadband source such as an incandescent light, a gas light, the sun, a laser (or an array of lasers with different central frequencies so to cover a broad spectrum), or a light emitting diode (LED). The metallic layer may further include a chemically sensitive layer (e.g., including or coated with molecules 737) that changes the plasmon resonance to a second electromagnetic resonance in the presence of target analyte 735. In some embodiments, chip 732 includes a layer of nanometallic particles configured to change a plasmon resonance to the electromagnetic radiation localized within the size of the nanometallic particles, in the presence of target analyte 735. In some embodiments, chip 732 includes a functional layer or chemically sensitive layer that may include an immunoassay, an RNA or DNA assay, or a nucleotide string selected to match the ribonucleic string of a pathogen (e.g., target analyte 735).

FIG. 7A is a diagram illustrating steps for collecting and processing a bio-measurement, according to some embodiments. A sample 702 is interacted 704 with a pre-selected molecule or ligand acting as a biorecognition element 712 such as an enzyme, an antibody (e.g., immunoassay), a protein, a nucleic acid, a receptor, a cell, and the like. A chip 732 interacted with sample 702 is interrogated optically by interacting with a first electromagnetic radiation via an optical transducer 706 based on techniques 714 such as a surface plasmon resonance (SPR), a localized SPR (LSPR), interferometry, resonance, grating dispersion, refractometry, and the like, to obtain a signal output 708 from a processor (e.g., in the IEM, or mobile device, or a computer coupled thereof). The signal output 708 is processed by computer 710, to arrive at a qualitative and/or quantitative determination of the presence of target analyte 735 in the sample.

FIG. 7B illustrates a layer of functional molecules 737 adhered to a gold coating on top of a glass cover. Functional layer 737 includes a ligand molecule that is chemically affine with target analyte 735. A fluidics system 730 in contact with functional layer 737 may carry a sample fluid (e.g., ear emissions, or in-ear gas) whereby ligand molecules 737 capture and immobilize target molecules 735, forming a ligand-target complex that alters the optical properties of chip 732 (e.g., an effective refraction index). For example, a change in effective refraction index may alter a Brewster angle for polarized radiation reflected off of the glass cover adjacent to a prism 727. A typical curve 752B of the signal over time starts from a baseline and grows during ligand-target association until an equilibrium is reached. In some embodiments, curve 752B may show a degree of dissociation until a new equilibrium point is reached. For performing a new measurement, a regeneration step includes removing the ligand-target complexes (e.g., by physical/chemical dissociation), to recover the baseline. The steps illustrated in the time-evolution of the signal may be calibrated into the detector so that at any point in time it may be possible to assess the concentration of a target analyte in the sample fluid.

FIG. 7C illustrates chip 732 configured as a two-dimensional (2D) imaging array of biorecognition elements wherein prism 727 couples (via total internal reflection and evanescent wave coupling) a first electromagnetic radiation from emitter 721, and a second electromagnetic radiation out into detector 723. The first electromagnetic radiation is a scanning beam sequentially reading each biorecognition element. In some embodiments, the first electromagnetic radiation may be a wide illumination beam, and a second electromagnetic radiation may be reflected or scattered off chip 732 and collected via imaging optics to form an image 752C of the chip wherein different colors indicate different levels of ligand-target absorption. Accordingly, image 752C may provide a spatial distribution of ear emissions, indicative of a localized health condition of the user.

FIG. 7D illustrates an optical sensor 700D wherein electromagnetic radiation 741 from emitter 721 (e.g., a laser) propagates through a planar waveguide 732D (e.g., via total internal reflection). An outer surface of the planar waveguide is in contact with a sample fluid 733D that contains target analytes 735 and antibodies 737D configured to couple to target analyte 735 with air 734 on the opposite side of planar waveguide 732D. Antibodies 737D may also be attached to a fluorescent dye 739. When target-analyte complex forms, it may precipitate or adhere (e.g., physically or chemically) to the outer surface of planar waveguide 732D. An electromagnetic radiation 742 includes sensible fluorescence emission excited by evanescent wave coupling of electromagnetic radiation 741 and fluorescent dye 739 on the outer surface of waveguide 732D. The amount of electromagnetic radiation 742 detected (e.g., via a detector or an imaging camera) may be correlated to the concentration of target analyte 735 in sample fluid 733D.

FIG. 7E illustrates a functional layer 737 including metal-organic nanoparticle complexes distributed across a 2D surface 732E, thus providing an LSPR signal in a transmissive mode 700E-1 and also in a reflectance mode 700E-2. Emitter 721 generates a first electromagnetic radiation 741, and a detector 723 receives a second electromagnetic radiation 742 (transmitted or reflected).

FIG. 7F illustrates a functional layer 700E-1 of molecules 737 on a planar substrate 732F-1, exposed to a fluid 733F that contains target analyte 735. An evanescent radiation 741F excites fluorescent emission from the ligand-target complex, which is then detected and quantified. In some embodiments, a functional layer 700E-2 over planar substrate 732F-2 may include nanoparticles 734 having ligand molecules 737 adhered to their surface (thus affording a high concentration in a small volume). The fluorescence emission of ligand-target complexes is then measured and quantified to determine a concentration of target analyte 735 in fluid 733F.

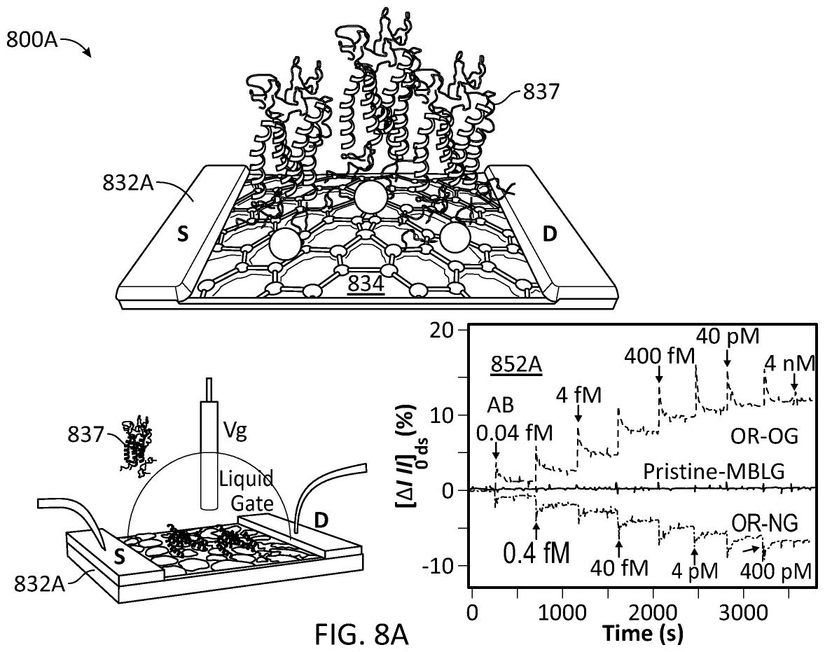

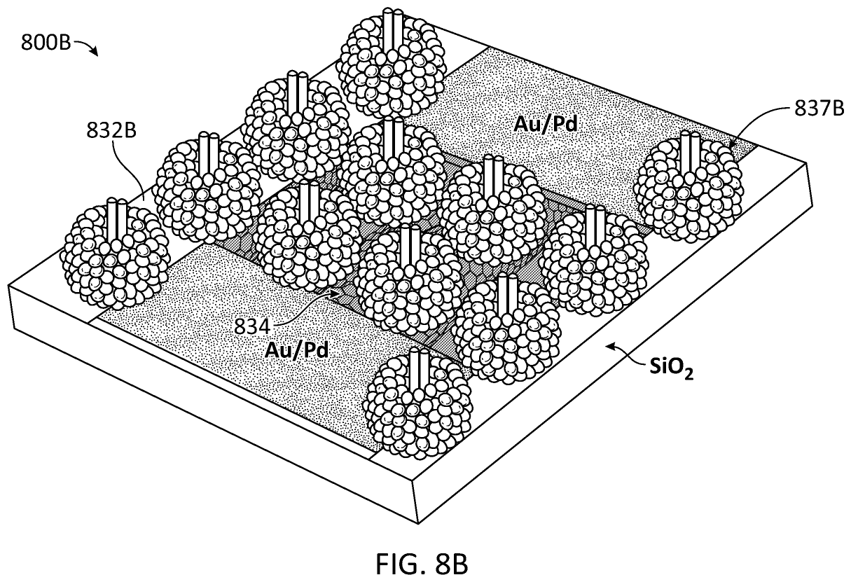

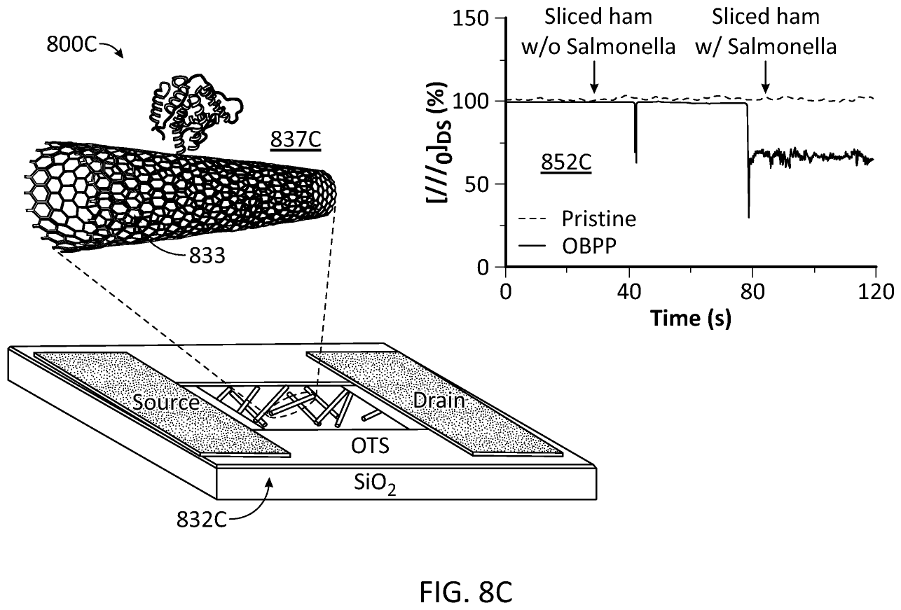

FIGS. 8A-8C illustrate several embodiments for chemical measurement sensors 800A, 800B, and 800C (hereinafter, collectively referred to as “chemical sensors 800”), in an IEM based on surface plasmon resonance, according to some embodiments. Chemical measurement sensors 800 may be used to identify trace amounts of gases and vapors resembling mammalian olfaction organs. In some embodiments, chemical sensors 800 make use of proteins 837 expressed in olfactory neuronal receptors (OR) in mammals. Accordingly, proteins 837 become charge carriers in the presence of certain “odorant” molecules (e.g., target analytes) via activation of chemical potentials altering ligand affinities in the protein structure. Thus, when proteins 837 are placed across a source-drain bridge in a transistor type architecture 832A or 832B (hereinafter, collectively referred to as “transistor structures 832”), a current is transmitted based on a level of activation of proteins 837 over a conductive graphene layer 834. Thus, current levels 852A and 852C (hereinafter, collectively referred to as “current levels 852”) across transistor structures 832 is indicative of a trace concentration of the “odor” generating molecule.

FIG. 8A illustrates a liquid-ion-gated field-effect-transistor (FET) chip 832A using an OR-conjugated modified bilayer graphene (MBLG) 834, according to some embodiments. In the figure, the signal waveform in current level 852A is shown to increase by discrete amounts as the concentration of the odorant molecule is increased, in solution (amyl butyrate (AB)). The signal is compared with a pristine MBLG FET.

FIG. 8B illustrates a chemical sensor 800B including immobilized nanovesicles 837B forming a single-walled nanotube (SWNT) FET 832B, according to some embodiments. Nanovesicles 837B are configured to form an ion channel (e.g., for Ca²⁺ or Na⁺) in the presence of the odorant molecule, and thus provide a charge channel for FET 832B.

FIG. 8C illustrates chemical sensor 800C with carbon nanotubes (CNT) FET 833 functionalized with an odorant binding protein-derived peptide (OBPP) 837C. Peptide 837C may be immobilized on an SiO₂ substrate via π-π interactions between Phenylalanine residues and the CNT. Curves 852C illustrate signal waveforms for detection of a bacterium (e.g., Salmonella) contamination on mammal tissue (e.g., a piece of sliced ham). The binding of Salmonella notably reduces the current load in FET 832C due to the increased impedance of the cellular membrane of the bacterium (e.g., as compared to the pristine sample).

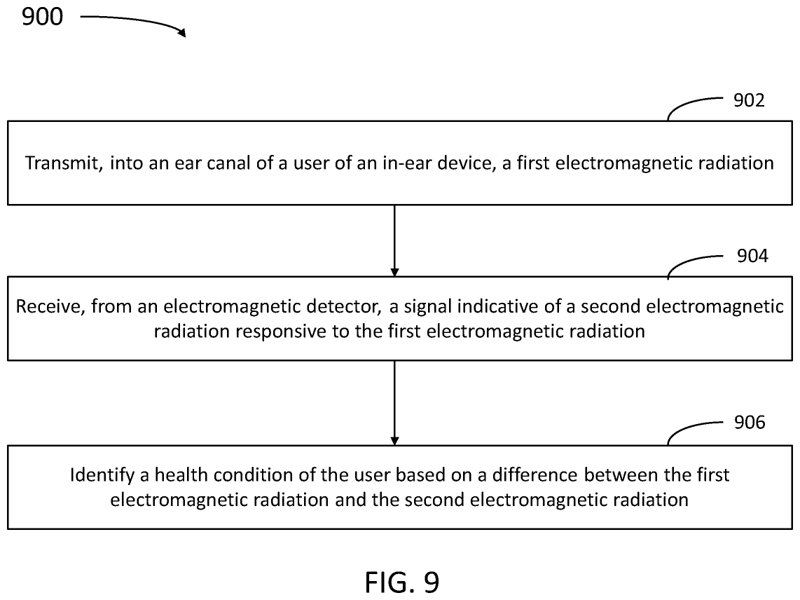

FIG. 9 is a flow chart illustrating steps in a method 900 for using optical sensors in an in-ear monitor for assessing the health of a user of a headset or smart glasses, according to some embodiments. In some embodiments, at least one or more of the steps in method 900 may be performed by a processor executing instructions stored in a memory in either one of smart glasses or other wearable device on a user's body part (e.g., head, arm, wrist, leg, ankle, finger, toe, knee, shoulder, chest, back, and the like). In some embodiments, at least one or more of the steps in method 900 may be performed by a processor executing instructions stored in a memory, wherein either the processor or the memory, or both, are part of a mobile device for the user, a remote server or a database, communicatively coupled with each other via a network (cf., processors 112, 312, and memory 120, client devices 110, server 130, and network 150). Moreover, the mobile device, the smart glasses, and the wearable devices may be communicatively coupled with each other via a wireless communication system and protocol (e.g., communications module 118, radio, Wi-Fi, Bluetooth, near-field communication—NFC—and the like). In some embodiments, a method consistent with the present disclosure may include one or more steps from method 900 performed in any order, simultaneously, quasi-simultaneously, or overlapping in time.

Step 902 includes transmitting, into an ear canal of a user of an in-ear device, a first electromagnetic radiation.

Step 904 includes receiving, from an electromagnetic detector, a signal indicative of a second electromagnetic radiation responsive to the first electromagnetic radiation. In some embodiments, the second electromagnetic radiation is indicative of a change in an optical property of a functional layer in a chip embedded in the in-ear device, and step 904 includes determining a presence of a pre-selected target substance based on the change in the optical property of the functional layer, wherein the health condition is correlated with the presence of the pre-selected target substance. In some embodiments, the first electromagnetic radiation includes a time-multiplex code, and step 904 includes decoding the signal according to the time-multiplex code.

Step 906 includes identifying a health condition of the user based on a difference between the first electromagnetic radiation and the second electromagnetic radiation. In some embodiments, the difference between the first electromagnetic radiation and the second electromagnetic radiation is a coherent phase difference, and step 906 includes interfering the first electromagnetic radiation with the second electromagnetic radiation. In some embodiments, step 906 includes determining a tissue displacement, density, or composition based on the coherent phase difference between the first and second electromagnetic radiation. In some embodiments, the second electromagnetic radiation includes a backscattered portion of the first electromagnetic radiation and step 906 includes identifying a cardio-respiratory condition based on a waveform of the backscattered portion of the first electromagnetic radiation. In some embodiments, a difference between the first electromagnetic radiation and the second electromagnetic radiation is indicative of a trace amount of a selected molecule in the ear canal of the user and step 906 includes determining that a concentration of the selected molecule is higher than a healthy threshold value. In some embodiments, the first electromagnetic radiation is in resonance with a plasmon mode of a metallic layer disposed in the in-ear device, and step 906 includes determining a presence of a pre-selected target substance based on a change of plasmon resonance to the second electromagnetic radiation.

### Hardware Overview

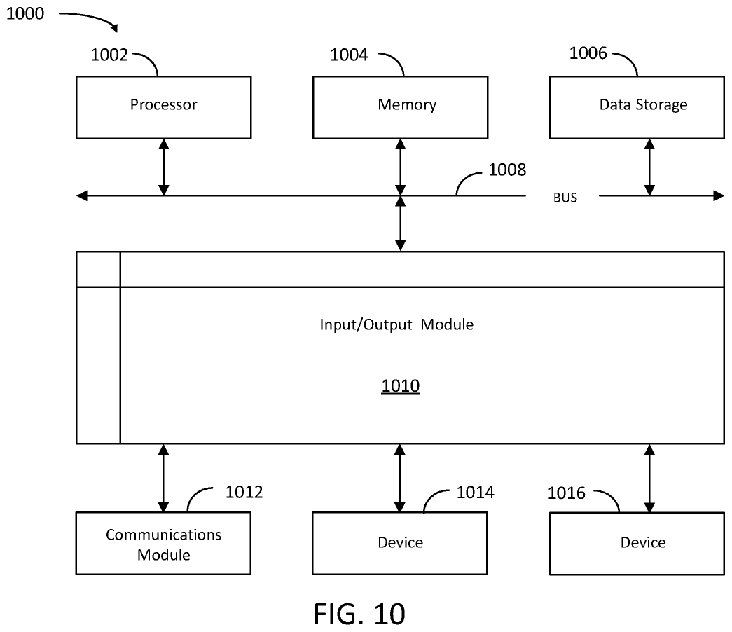

FIG. 10 is a block diagram illustrating an exemplary computer system 1000 with which headsets and other client devices 110, and method 900 can be implemented, according to some embodiments. In certain aspects, computer system 1000 may be implemented using hardware or a combination of software and hardware, either in a dedicated server, or integrated into another entity, or distributed across multiple entities. Computer system 1000 may include a desktop computer, a laptop computer, a tablet, a phablet, a smartphone, a feature phone, a server computer, or otherwise. A server computer may be located remotely in a data center or be stored locally.

Computer system 1000 includes a bus 1008 or other communication mechanism for communicating information, and a processor 1002 (e.g., processors 112) coupled with bus 1008 for processing information. By way of example, the computer system 1000 may be implemented with one or more processors 1002. Processor 1002 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1000 can include, in addition to hardware, code that creates an execution environment for the computer program in question, e.g., code that constitutes processor firmware, a protocol stack, a database management system, an operating system, or a combination of one or more of them stored in an included memory 1004 (e.g., memory 120), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 1008 for storing information and instructions to be executed by processor 1002. The processor 1002 and the memory 1004 can be supplemented by, or incorporated in, special purpose logic circuitry.

The instructions may be stored in the memory 1004 and implemented in one or more computer program products, e.g., one or more modules of computer program instructions encoded on a computer-readable medium for execution by, or to control the operation of, the computer system 1000, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1004 may also be used for storing temporary variable or other intermediate information during execution of instructions to be executed by processor 1002.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1000 further includes a data storage device 1006 such as a magnetic disk or optical disk, coupled with bus 1008 for storing information and instructions. Computer system 1000 may be coupled via input/output module 1010 to various devices. Input/output module 1010 can be any input/output module. Exemplary input/output modules 1010 include data ports such as USB ports. The input/output module 1010 is configured to connect to a communications module 1012. Exemplary communications modules 1012 include networking interface cards, such as Ethernet cards and modems. In certain aspects, input/output module 1010 is configured to connect to a plurality of devices, such as an input device 1014 and/or an output device 1016. Exemplary input devices 1014 include a keyboard and a pointing device, e.g., a mouse or a trackball, by which a consumer can provide input to the computer system 1000. Other kinds of input devices 1014 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 1016 include display devices, such as an LCD (liquid crystal display) monitor, for displaying information to the consumer.

According to one aspect of the present disclosure, headsets and client devices 110 can be implemented, at least partially, using a computer system 1000 in response to processor 1002 executing one or more sequences of one or more instructions contained in memory 1004. Such instructions may be read into memory 1004 from another machine-readable medium, such as data storage device 1006. Execution of the sequences of instructions contained in main memory 1004 causes processor 1002 to perform the process steps described herein. One or more processors in a multi-processing arrangement may also be employed to execute the sequences of instructions contained in memory 1004. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical consumer interface or a Web browser through which a consumer can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1000 can include clients and servers. A client and server are generally remote from each other and typically interact through a communication network. The relationship of client and server arises by virtue of computer programs running on the respective computers and having a client-server relationship to each other. Computer system 1000 can be, for example, and without limitation, a desktop computer, laptop computer, or tablet computer. Computer system 1000 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1002 for execution. Such a medium may take many forms, including, but not limited to, non-volatile media, volatile media, and transmission media. Non-volatile media include, for example, optical or magnetic disks, such as data storage device 1006. Volatile media include dynamic memory, such as memory 1004. Transmission media include coaxial cables, copper wire, and fiber optics, including the wires forming bus 1008. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A device, comprising:
an in-ear fixture configured to fit in an ear canal of a user;
an emitter mounted on the in-ear fixture and configured to emit a first electromagnetic radiation onto the ear canal of the user;
a detector configured to provide a signal indicative of a second electromagnetic radiation from the ear canal of the user; and
a processor that is coupled to an augmented reality headset, the processor configured to identify a health condition of the user based on the signal, wherein the second electromagnetic radiation includes at least a portion of the first electromagnetic radiation reflected from a tissue in the ear canal of the user.

2. The device of claim 1, wherein the first electromagnetic radiation includes one of a near-infrared or green light, and the health condition of the user includes a cardio-respiratory condition.

3. The device of claim 1, wherein a difference between the first electromagnetic radiation and the second electromagnetic radiation is indicative of a trace amount of a selected molecule in the ear canal of the user.

4. The device of claim 1, wherein a difference between the first electromagnetic radiation and the second electromagnetic radiation is indicative of a glucose content in a blood stream of the user.

5. The device of claim 1, further comprising a chip having a functional layer including a photochemical substance that changes an optical property in a presence of a pre-selected target substance, wherein the second electromagnetic radiation is indicative of a change in the optical property of the functional layer.

6. The device of claim 1, further comprising a chip having a metallic layer configured to form a plasmon resonance in response to the first electromagnetic radiation, wherein the metallic layer further includes a chemically sensitive layer that changes the plasmon resonance to the second electromagnetic radiation in a presence of a pre-selected target substance.

7. The device of claim 1, further comprising a chip having a layer of nanometallic particles configured to change a plasmon resonance to the second electromagnetic radiation localized within a size of a nanometallic particle in a presence of a pre-selected target substance.

8. The device of claim 1, further comprising an electrode mounted on the in-ear fixture, and configured to receive an electronic signal indicative of a cardio-respiratory activity of the user, and the processor is configured to identify the health condition of the user based on a correlation of the signal with the electronic signal.

9. The device of claim 1, wherein the emitter includes a pulsed radiation source, and the processor is configured to filter the signal from the second electromagnetic radiation according to the pulsed radiation source.

10. The device of claim 1, further comprising a thin film filter to adjust a spectral bandwidth of the first electromagnetic radiation or the second electromagnetic radiation.

11. A system, comprising:
a memory storing multiple instructions; and
one or more processors configured to execute the instructions and cause the system to perform operations, comprising:
to transmit, into an ear canal of a user of an in-ear device, a first electromagnetic radiation;
to receive, from an electromagnetic detector, a signal indicative of a second electromagnetic radiation responsive to the first electromagnetic radiation; and
to identify a health condition of the user based on a difference between the first electromagnetic radiation and the second electromagnetic radiation.

12. The system of claim 11, wherein the first electromagnetic radiation includes one of a near-infrared light, a red light, or a green light and to identify a health condition the one or more processors execute instructions to associate a difference between the first electromagnetic radiation and the second electromagnetic radiation with a blood-oxygenation level.

13. The system of claim 11, wherein the first electromagnetic radiation and the second electromagnetic radiation have bandwidth within a glucose absorption band.

14. The system of claim 11, wherein the first electromagnetic radiation is modulated with a time-multiplexing code, and to determine the difference between the first electromagnetic radiation and the second electromagnetic radiation the one or more processors execute instructions to demodulate the signal with the time-multiplexing code.

15. A computer-implemented method, comprising:
transmitting, into an ear canal of a user of an in-ear device, a first electromagnetic radiation;
receiving, from an electromagnetic detector, a signal indicative of a second electromagnetic radiation responsive to the first electromagnetic radiation; and
identifying a health condition of the user based on a difference between the first electromagnetic radiation and the second electromagnetic radiation.

16. The computer-implemented method of claim 15, wherein the second electromagnetic radiation is indicative of a change in an optical property of a functional layer in a chip embedded in the in-ear device, further comprising determining a presence of a pre-selected target substance based on the change in the optical property of the functional layer, wherein the health condition is correlated with the presence of the pre-selected target substance.

17. The computer-implemented method of claim 15, wherein the first electromagnetic radiation includes a time-multiplex code, and receiving a signal indicative of the second electromagnetic radiation comprises decoding the signal according to the time-multiplex code.

18. The computer-implemented method of claim 15, wherein the second electromagnetic radiation includes a backscattered portion of the first electromagnetic radiation and identifying a health condition of the user includes identifying a cardio-respiratory condition based on a waveform of the backscattered portion of the first electromagnetic radiation.

19. The computer-implemented method of claim 15, wherein a difference between the first electromagnetic radiation and the second electromagnetic radiation is indicative of a trace amount of a selected molecule in the ear canal of the user and identifying health condition of the user includes determining that a concentration of the selected molecule is higher than a healthy threshold value.

20. The computer-implemented method of claim 15, wherein the first electromagnetic radiation is in resonance with a plasmon mode of a metallic layer disposed in the in-ear device, and wherein identifying a health condition of the user comprises determining a presence of a pre-selected target substance based on a change of plasmon resonance to the second electromagnetic radiation.
